(19) United States
(12) Patent Application Publication
(10) Pub. No.: US 2009/0304820 A1
(43) Pub. Date: Dec. 10, 2009

(54) SOLUBILIZED BENZOYL PEROXIDE TOPICAL DRUG FORMULATION FOR THE TREATMENT OF ACNE

(76) Inventor: Pankaj Modi, Ancaster (CA)

Correspondence Address: HEENAN BLAIKIE LLP, BAY ADELAIDE CENTRE, 333 BAY STREET, SUITE 2900, P.O. BOX 2900, TORONTO, ON, M5H 2T4, CA

(21) Appl. No.: 12/133,939

(22) Filed: Jun. 5, 2008

(51) Int. Cl. A61K 33/00; A61K 31/327; A61P 17/10; A61K 31/60

(52) U.S. Cl. 424/722; 514/714; 514/165

(57) ABSTRACT

The invention relates to a novel solubilized benzoyl peroxide topical formulation for the treatment of acne comprising: benzoyl peroxide, one or more skin penetration enhancers, a surfactant, and one or more solvents, wherein the benzoyl peroxide is solubilized in the solvent. The invention further relates to the use of the topical formulation as well as a process for making the topical formulation.

### FIELD

The present invention relates to a solubilized benzoyl peroxide topical formulation for the treatment of acne.

### BACKGROUND

Acne vulgaris (“acne”) is a disorder resulting from the action of hormones on the skin's oil glands (sebaceous glands), wherein the sebaceous glands of the skin produce excess sebum, and become enlarged and/or infected when the pore opening becomes plugged with a comedo (a mixture of keratin and sebum). The symptoms of acne include plugged pores and outbreaks of inflamed lesions commonly called pimples.

Acne lesions usually occur on the face, neck, back, chest, and shoulders. It is the most common skin disease amongst teenagers and young adults. Acne can occur at any age, and is common to all ethnic backgrounds. Nearly 85 percent of people between the ages of 12 and 24 develop this disorder. For most people, acne tends to go away by the time they reach their thirties. However, some people in their forties and fifties continue to have acne, commonly termed “adult acne”.

Several acne treatments are commercially available, such as those in the following categories, for example: topical bactericidals (e.g. triclosan, chlorhexidine gluconate and benzoyl peroxide); topical antibiotics (e.g. erythromycin, tetracycline and clindamycin); oral antibiotics (e.g. tetracyclines and trimethoprim); hormonal treatments (e.g. hormonal contraception in females); topical retinoids (e.g. tretinoin, adapalene and tazarotene); oral retinoids (e.g. isotretinoin, marketed as Accutane®, Roche Pharmaceuticals, New Jersey); and phototherapy.

Many of the over-the-counter (“OTC”) acne medications currently marketed rely on chemicals which have an antibacterial and/or a peeling/drying action which aids in breaking down keratin (i.e. keratolytic agent), thus helping to clear plugged pores.

Consequently, there are a wide variety of drugs that are used in topical formulations for the treatment of acne, and there are disadvantages in respect of each drug or combination of drugs. Benzoyl peroxide, resorcinol, salicylic acid, and sulfur are among the most common topical OTC agents used to treat acne. Benzoyl peroxide has an antibacterial effect and also acts as a peeling and drying agent, increasing cell turnover and helping to clear plugged pores.

In general, topical formulations used to treat acne must be water-based, as oil-based formulations can cause or further aggravate acne eruptions.

Benzoyl peroxide is a crystalline solid with a melting point of 103° C. to 106° C., and is insoluble in water.

Benzoyl peroxide is soluble in organic solvents, but most organic solvents are toxic and therefore not suitable for pharmaceutical/cosmetic use. Polar organic solvents such as acetone and ethanol are less toxic, but the flammability of these solvents as well as the ability of these solvents to irritate and strip the skin of its protective mantle limit their use in pharmaceutical or cosmetic compositions.

A common method of treating acne vulgaris is to treat the skin with a benzoyl peroxide dispersion of crystals in a gel/lotion/cream base. However, the use of essentially undissolved benzoyl peroxide crystals has limited efficacy, and consequently requires high concentrations of benzoyl peroxide to be effective. Furthermore, it is difficult for the benzoyl peroxide crystals to penetrate into the comedone and into the sebaceous gland itself, because the comedone plug is a physical barrier and the size of the follicular opening is limited.

Consequently, commercially available topical formulations of benzoyl peroxide have several disadvantages, since they: (1) have a limited ability to penetrate the skin and consequently have low efficacy for the dose administered; (2) cannot be formulated as clear compositions and sprays; and (3) leave an aesthetically undesirable white, powder residue on the skin once the topical formulation dries.

### SUMMARY

In one aspect, this patent application discloses a solubilized benzoyl peroxide topical formulation that comprises benzoyl peroxide, one or more solvents, one or more skin penetration enhancers, and a surfactant.

The solubilized benzoyl peroxide topical formulation may comprise one or more solvents that are selected from ethylene glycol-400 (low molecular weight solvent), propylene or butylene glycol, isopropyl or ethyl alcohol, short chain alkyl esters, or combinations thereof.

The solubilized benzoyl peroxide topical formulation may comprise the surfactant sodium lauryl sulfate. The solubilized benzoyl peroxide topical formulation may comprise the skin penetration enhancer dimethylsulfoxide (DMSO).

In a preferred embodiment, the solubilized benzoyl peroxide topical fonnulation comprises: benzoyl peroxide, the skin penetration enhancer dimethylsulfoxide (DMSO), the surfactant sodium lauryl sulfate and the solvents: ethylene glycol-400, propylene or butylene glycol and isopropyl or ethyl alcohol.

In another embodiment, this patent application discloses the use of the solubilized benzoyl peroxide topical formulation for the treatment and/or prophylaxis of acne.

In a further embodiment, this patent application discloses a process for the preparation of the solubilized benzoyl peroxide topical formulation.

The solubilized benzoyl peroxide topical formulation may include additional ingredients to form an emulsion, suspension, cream, lotion, gel or stick for topical administration.

### BRIEF DESCRIPTION OF THE DRAWINGS

Embodiments of the invention will be described, by way of example only, with reference to the appended drawings, wherein:

FIG. 1 shows a prior art unsolubilized benzoyl peroxide (gritty topical formulation) on human skin at a magnification of 500×;

FIG. 2 shows the solubilized benzoyl peroxide topical formulation on human skin at a magnification of 500×; and

FIGS. 3 to 6 are photographs of the facial skin of individual patients who participated in a clinical trial of the solubilized benzoyl peroxide topical formulation.

### DETAILED DESCRIPTION

The present invention generally provides for the manufacture and use of formulations of solubilized benzoyl peroxide. The solubilized benzoyl peroxide formulations provide a solvent vehicle for the treatment of acne and comprise solubilized benzoyl peroxide molecules that are available to penetrate into follicles, follicle oil glands, the stratum corneum and the epidermis of the skin. The solubilized benzoyl peroxide saturates the targeted infected areas to destroy the bacteria-causing acne.

The solubilized benzoyl peroxide topical formulation comprises: benzoyl peroxide, a solvent, a skin penetration enhancer, and a surfactant.

### Benzoyl Peroxide

Benzoyl peroxide is normally commercially available as either pure crystals or in a wet crystalline state. Either of these or other forms of benzoyl peroxide can be mixed with the solvents described below to form the solubilized benzoyl peroxide topical formulation.

Due to the increased skin penetration of the benzoyl peroxide of the solubilized benzoyl peroxide topical formulation, the minimum effective amount of benzoyl peroxide can be lower than the amount of benzoyl peroxide used in presently available acne formulations containing benzoyl peroxide.

Thus, for example, a solubilized benzoyl peroxide topical formulation containing 5% benzoyl peroxide has 2 to 3 times the skin penetration than prior art 10% benzoyl peroxide compositions.

The amount of benzoyl peroxide solubilized in the solvent will vary depending on a number of factors, including, for example, the desired activity of benzoyl peroxide, the ultimate form of the product, and the particular solvent employed. The benzoyl peroxide will constitute from 0.5 to 70% benzoyl peroxide by weight, preferably from 1 to 30% benzoyl peroxide by weight, more preferably from 1 to 10% benzoyl peroxide by weight, and most preferably from 2 to 5% benzoyl peroxide by weight of the solubilized benzoyl peroxide topical formulation.

### Solvents

Solvents useful for solubilizing benzoyl peroxide and preparing the solubilized benzoyl peroxide topical formulation include, alone or in combination, for example: ethylene glycol-400 (low molecular weight solvent), propylene or butylene glycol, isopropyl or ethyl alcohol, short chain alkyl esters, ethers, aldehydes, ketones or alcohols of benzoic acid, benzyl alcohol, phenol or phathalic acid, aryl esters, ethers, alcohols of benzoic acid, benzyl alcohol, phenol, alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol, alkyl ethers of phenol, benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzyl alcohol, ethyl benzyl alcohol, phenyl benzoate, phenyl ether, benzyl benzoate, and phenyl ethyl ester.

Preferred solvents are ethylene glycol-400 (low molecular weight solvent), propylene or butylene glycol, isopropyl or ethyl alcohol, and short chain alkyl esters.

The amount of solvent used to solubilize the benzoyl peroxide will vary depending on a number of factors, including, for example, the ultimate form of the product and the particular solvent employed. Generally, the solvent will constitute from 1 to 70 percent, by weight, of the solubilized benzoyl peroxide topical formulation.

### Skin Penetration Enhancers

Skin penetration enhancers promote the absorption of an active ingredient by the skin. One or more skin penetration enhancers may be used to facilitate the permeation of benzoyl peroxide through the patient's skin.

Examples of skin penetration enhancers include, but are not limited to, dimethylsulfoxide (DMSO), alcohols (such as short chain alcohols, long chain alcohols, or polyalcohols), amines and amides (such as urea, amino acids or their esters), AZONE(R) (including derivatives of AZONE(R)), pyrrolidones (including derivatives of pyrrolidones), terpenes (including derivatives of terpenes), fatty acids and their esters, macrocyclic compounds, tensides, sulfoxides (including decylmethylsulfoxide), liposomes, micelles, transfersome, lecithin vehicles, ethosomes, surfactants (such as anionic, cationic, and nonionic surfactants), essential oils, d-limonene, Quillaja saponaria (QTS), Acanthophyllum squarrusom (ATS), allantoin, fulvic acid, myrrh, Eldopaque and/or hydroquinone glyquin

A preferred skin penetration enhancer is DMSO. The concentration of DMSO can range from 0.5 to 8%, and preferably from 1 to 4% by weight of the solubilized benzoyl peroxide topical formulation.

### Surfactants

The solubilized benzoyl peroxide topical formulation also contains one or more surfactants.

Suitable surfactants include both naturally occurring compounds as well as synthetic surfactants. Examples of suitable surfactants include: phospholipids and cholates, polysorbates (i.e. fatty acid esters of polyethoxylated sorbitol), polyethylene glycol esters of fatty acids from sources such as castor oil, polyethoxylated fatty acids (e.g. stearic acid), octylphenolpoly(ethyleneglycolether), polyethoxylated isooctylphenol/formaldehyde polymer, poloxamers (e.g. poly(oxyethylene)-poly(oxypropylene)block copolymers), polyoxyethylene fatty alcohol ethers, polyoxyethylene nonylphenyl ethers, polyoxyethylene isooctylphenyl ethers, SDS, phospholipids (e.g. phosphatidylcholines (lecithins), including soy or egg lecithin), phosphatidylglycerol, phosphatidylinositol, phosphatidylserine, phosphatidic acid, cardiolipin, and phosphatidylethanolamine.

Mixtures of surfactant molecules, including mixtures of surfactants of different chemical types, are acceptable. Surfactants should be suitable for cosmetic or pharmaceutical administration and compatible with benzoyl peroxide.

A preferred surfactant is sodium lauryl sulfate. The concentration of sodium lauryl sulfate can range from 0.5 to 8%, and preferably from 1 to 4% by weight of the solubilized benzoyl peroxide topical formulation.

### Additional Components

Various other ingredients can optionally be included in the solubilized benzoyl peroxide topical formulation, such as: topical anesthetics (e.g. benzocaine, lidocaine, tetracaine, prilocaine), antibiotics/antimicrobials/bactericidals (including dermatologically acceptable salts of tetracylin and tetracyclin derivatives, gentamycin, kanamycin, streptomycin, neomycin, capreomycin, lineomycin, paromomycin, tobramycin, erythromycin, triclosan, antimicrobial peptides, octopirox, parachlorometa xylenol nystatin, tolnaftate, miconazole hydrochloride, chlorhexidine gluconate, chlorhexidin hydrochloride, methanamine hippurate, methanamine mandelate, minocycline hydrochloride, clindamycin, cleocin, β-lactam derivatives such as aminopenicillin and mixtures thereof), 1 to 2% sodium hydroxide, salicylic acid, and other medicinal ingredients in amounts effective for the treatment of acne.

The solubilized benzoyl peroxide topical formulation can be added to other ingredients to form desired products, including: serums, toners, pumps or aerosol sprays, clear gels, sticks, creams, lotions and mousses, solutions, emulsions (including microemulsions), suspensions, creams, lotions, gels, sticks, powders, or other typical solid or liquid compositions used for treatment of skin. Such compositions may also contain cooling, solvent constituents and other ingredients typically used in such products, such as moisturizers and hydration agents, preservatives, emulsifiers, natural or synthetic oils, surfactants, detergents, gelling agents, emollients, antioxidants, fragrances, fillers, thickeners, waxes, odor absorbers, dyestuffs, coloring agents, powders, viscosity-controlling agents and water, and optionally including anti-itch actives, botanical extracts, conditioning agents, darkening or lightening agents, glitter, humectants, mica, minerals, polyphenols, silicones or derivatives thereof, sun blocks, vitamins, and phytomedicinals, and combinations thereof, for example.

When preparing desired products (e.g., emulsions, lotions, creams or gels) the solubilized benzoyl peroxide topical formulation can be added to other ingredients to form desired products at low temperatures (e.g. 25 to 40° C.). In these processes, since benzoyl peroxide is never in contact with substantial heat, the possibility of decomposition or fire is greatly reduced.

Suitable suspending agents include the following constituents, for example: polyacrylamide, C13-14 isoparafin & laureth 7; C13-14 isoparaffin, mineral oil, polyacrylate, polyacrylamide and ethoxylated sorbitan ester; acrylamide/sodium acryloyldimethyl taurate copolymer, isohexadecane and ethoxylated sorbitan ester; and combinations thereof.

In some emulsions, the aqueous phase constituting the dispersion medium may include any suitable surfactant, humectant, suspending agent, and/or buffer systems, and combinations thereof suitable for combining with benzoyl peroxide. Examples of suitable humectants include glycerin; however any material capable of obtaining moisture may be added provided it is stable with benzoyl peroxide.

### Preparation of the Solubilized Benzoyl Peroxide Topical Formulation

The solubilized benzoyl peroxide topical formulation is prepared by combining the one or more solvents, one or more skin penetration enhancers, and surfactant and stirring at a temperature between 25 to 40° C. The combination of these ingredients results in the formation of micelles. Benzoyl peroxide is then added to the solution with continuous stirring to form the solubilized benzoyl peroxide topical formulation.

The solubility of benzoyl peroxide in the solvent(s) offers an improved method for preparing anhydrous benzoyl peroxide without subjecting the composition to any heat during processing. For example, when benzoyl peroxide-wet crystals containing 25% water are mixed with one or more solvents, the solvents (which solubilize the benzoyl peroxide) replace water in the process of changing the crystalline benzoyl peroxide into a solution, and the water can be readily separated.

In contrast to prior art formulations of benzoyl peroxide, the benzoyl peroxide will actively go into solution at levels as high as 10% by weight of the solubilized benzoyl peroxide topical formulation. Furthermore, the solubilized benzoyl peroxide topical formulation is translucent and has increased efficacy.

If levels of benzoyl peroxide are desired that exceed the solubility parameters of the solvent(s), then a saturated solution of a fine soft benzoyl peroxide slurry is formed. This composition can then be filtered to remove the water from the composition, thereby providing a fine textured, substantially water reduced benzoyl peroxide paste/saturated solution composition.

The examples which follow are intended to illustrate specific embodiments of the invention.

### EXAMPLE 1

### Formulation Method

The following solvents and additional ingredients are combined:
100 mL Ethylene Glycol-400 (low molecular weight solvent)
100 mL Propylene or Butylene Glycol
10-20 mL Isopropyl or Ethyl Alcohol
1% Sodium Lauryl Sulfate
1% DMSO
1% NaOH solution

Heat the above solution to 30-35° C. with constant stirring.

Add Benzoyl Peroxide powder (5% by wt or 3.5% by wt).

The following components can also be added, if desired: 2-8% wt of colloidal sulfur, 2% salicylic acid, 2% resorcinol or phenol, 3% glycerin and 2% benzocaine and 2% tetracaine dissolved in 20 mL ethyl alcohol to the above solution at 30-35° C. and stir the solution for 45 minutes.

The result is a homogenous milky solution without any gritty feeling. Cool the solution to room temperature, and add 20 mL of hydrogen peroxide solution (3%). The following ingredients may be added as needed to produce a lotion: water (as needed), 20 mL (3%-5% hydrogen peroxide), glyceryl stearate, PEG-100 stearate, cetearyl alcohol, dimethicone, panthenol, allantoin, carbomer, ceteareth-20, xanthan gum, triethanolamine, fragrance (parfum), diazolidinyl urea, methylparaben, propylparaben.

The resultant emulsion or clear solution or gel has been found to be stable at room temperature for at least one year.

### EXAMPLE 2

### In-Vitro Percutaneous Absorption of Solublized Benzoyl Peroxide Topical Formulation

### Materials and Methods

Transdermal absorption of the solubilized benzoyl peroxide topical formulation of Example 1 containing radiolabelled benzoyl peroxide (using I-125) was measured over a 24 hour period in human cadaver skin using the Franz in vitro diffusion chamber.

A 5% benzoyl peroxide topical formulation of the invention was applied onto the human cadaver cell uniformly with gentle rubbing for 1 min. Every 3 hours the percent of benzoyl peroxide recovered in the stratum corneum, epidermis, and stratum corneum surface was determined by calculating the percent recovery of the total amount of benzoyl peroxide applied from the bottom of the diffusion chamber. The total micrograms of benzoyl peroxide recovered from each location was calculated by measuring the radioactivity counts using a scintillation counter. Over the 24 hour study period, approximately 77% of the benzoyl peroxide applied penetrated the skin as shown in Table 1 below.

TABLE 1
Total dose applied; 5 mg (5000 microgram) on 5 cm area

| | Percentage of |
|---|---|
| | Benzoyl Peroxide Recovered |
| Time | (based on radioactivity counts) |
| 0 | 0 |
| 2 | 7 |
| 4 | 11 |
| 7 | 15 |
| 10 | 33 |
| 15 | 54 |
| 20 | 67 |
| 22 | 77 |

### EXAMPLE 3

### A Comparison of Patterns of Deposition of Two Formulations of Benzoyl Peroxide on Skin: Scanning Electron Micrograph on Silicone Wafer×500

A dramatic change in the magnitude of benzoyl peroxide solubility occurred above a dielectric constant value of about 20. The solubility of this drug can be enhanced by the replacement of a polar solvent by a vehicle of lower dielectric constant. A stable submicron emulsion gel was made with cremophor EL, glycerol, caprilic-capric triglycerides, and water in the proportion of 20-20/35/25, respectively; 3.5% benzoyl peroxide was also added. This submicron emulsion vehicle consisted of oil droplets, with a mean diameter of approximately 100-150 nm, dispersed in a continuous water phase. These studies confirm the potential of benzoyl peroxide incorporation into a submicron emulsion gel and the stability of this formulation.

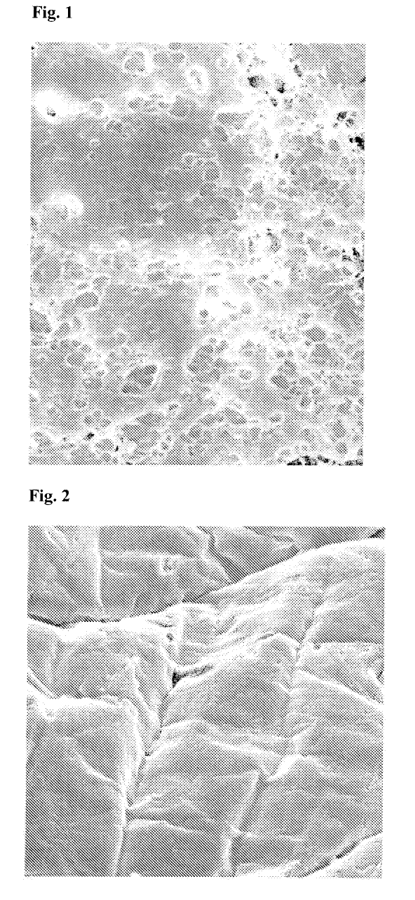

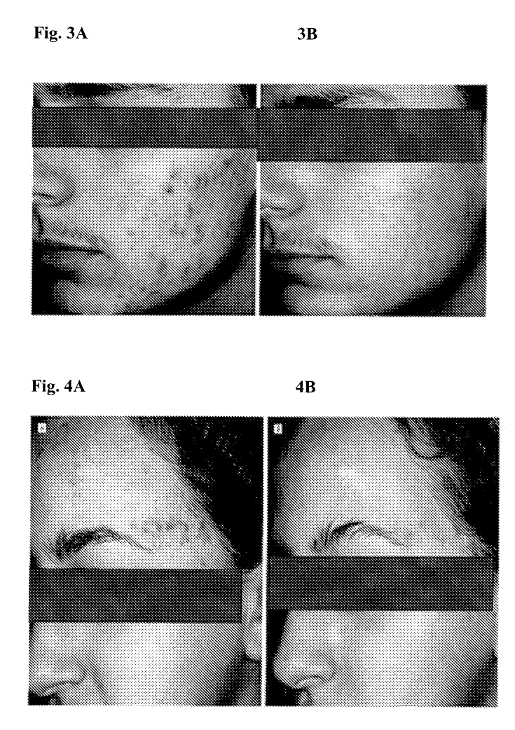

FIG. 1 shows a prior art unsolubilized benzoyl peroxide formulation (gritty formulation) on human skin at a magnification of 500×. FIG. 2 shows the solubilized benzoyl peroxide topical formulation of Example 1 on human skin at a magnification of 500×.

The results of this study demonstrated that the solubilized benzoyl peroxide topical formulation is distributed evenly over the surface of the skin, and lacks the gritty, granular appearance of the prior art benzoyl peroxide formulations.

### EXAMPLE 4

### Human Clinical Studies (Before and After Photographs)

Objective: To assess acne improvement and tolerability of the solubilized benzoyl peroxide topical formulation of Example 1 (at 3.5% benzoyl peroxide) during 12 weeks of treatment in comparison to a non-medicated cream control.

Participants: A total of 99 patients aged 12 to 39 years with facial acne were enrolled in the study.

Intervention: The study was randomized, and controlled by using a non-medicated (vehicle) cream identical to the base of the active cream. The medication samples were distributed in identical boxed pairs of 30 g tubes labeled “morning application” and “evening application”. The dosage of cream per application was approximately 0.6 g, described and demonstrated to patients as a pea-sized amount. Patients were requested to maintain diary records in which they recorded treatment periods of all applications. Clinical assistants collected these records at each visit. Patients were seen at baseline, defined as the visit when treatment was initiated, and again at 2, 4, 8, and 12 weeks of treatment.

Photography: During each visit, front and bilateral 45° side facial views of every patient were taken using a platform-mounted 35-mm SLR camera system (Nikon Corp, Tokyo, Japan) with a fixed-magnification 60-mm lens (f2.8) (Nikkor; Nikon Corp) and a dual-point light system (Twinflash; Canfield Scientific, Inc, Fairfield, N.J.). Patients were positioned in a stereotactic device designed to capture registered serial photographs using standardized subject angles, framing, lighting, exposure, and reproduction ratio.

Main Outcome Measures: Efficacy was based on reduction in acne lesions, treatment success (50%-100% improvement in global response to treatment) and improvement in overall disease severity.

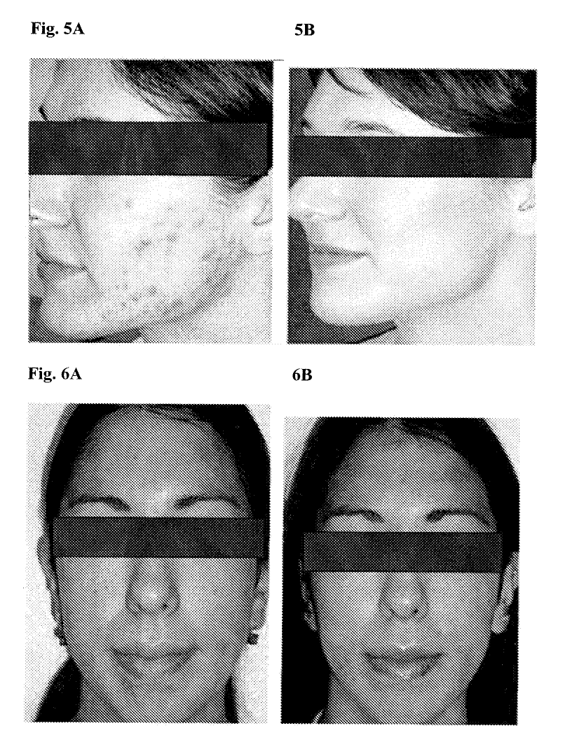

Results: FIGS. 3 to 6 are representative before and after treatment photos of study participants. FIGS. 3A, 4A, 5A and 6A are before treatment photos of study participants. FIGS. 3B, 4B, 5B and 6B are post-treatment photos of patients following 12 weeks of treatment with the topical formulation of Example 1 (at 3.5% benzoyl peroxide).

Conclusion: The 3.5% solubilized benzoyl peroxide topical formulation of Example 1 was found to be a safe and effective new method of acne treatment.

Numerous modifications, variations and adaptations may be made to the particular embodiments of the invention described above, without departing from the scope of the invention, which is defined in the following claims.

1. A solubilized benzoyl peroxide topical formulation comprising:
benzoyl peroxide;
one or more solvents;
one or more skin penetration enhancers; and
a surfactant.

2. The solubilized benzoyl peroxide topical formulation of claim 1 wherein the one or more solvents is selected from: ethylene glycol-400 (low molecular weight solvent), propylene or butylene glycol, isopropyl or ethyl alcohol, short chain alkyl esters, ethers, aldehydes, ketones or alcohols of benzoic acid, benzyl alcohol, phenol or phathalic acid, aryl esters, ethers, alcohols of benzoic acid, benzyl alcohol, phenol, alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol, alkyl ethers of phenol, benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzyl alcohol, ethyl benzyl alcohol, phenyl benzoate, phenyl ether, benzyl benzoate, and phenyl ethyl ester or combinations thereof.

3. The solubilized benzoyl peroxide topical formulation of claim 2 wherein the one or more solvents is selected from ethylene glycol-400 (low molecular weight solvent), propylene or butylene glycol, isopropyl or ethyl alcohol, short chain alkyl esters, or combinations thereof.

4. The solubilized benzoyl peroxide topical formulation of claim 3 wherein the skin penetration enhancer is dimethylsulfoxide (DMSO).

5. The solubilized benzoyl peroxide topical formulation of claim 4 wherein the surfactant is sodium lauryl sulfate.

6. The solubilized benzoyl peroxide topical formulation of claim 5 wherein the benzoyl peroxide is in a concentration of from 1 to 10% by weight.

7. The solubilized benzoyl peroxide topical formulation of claim 6 wherein the benzoyl peroxide is in a concentration of from 2 to 5% by weight.

8. The solubilized benzoyl peroxide topical formulation of claim 7 wherein the solubilized benzoyl peroxide topical formulation further comprises a topical anesthetic agent.

9. The solubilized benzoyl peroxide topical formulation of claims 7 wherein the solubilized benzoyl peroxide topical formulation further comprises an antibiotic/bactericidal/antibacterial agent.

10. The solubilized benzoyl peroxide topical formulation of claim 9 wherein the antibiotic/bactericidal/antibacterial agent is selected from: tetracylin and tetracyclin derivatives, gentamycin, kanamycin, streptomycin, neomycin, capreomycin, lineomycin, paromomycin, tobramycin, erythromycin, triclosan, antimicrobial peptides, octopirox, parachlorometa xylenol nystatin, tolnaftate, miconazole hydrochloride, chlorhexidine gluconate, chlorhexidin hydrochloride, methanamine hippurate, methanamine mandelate, minocycline hydrochloride, clindamycin, cleocin, β-lactam derivatives such as aminopenicillin and mixtures thereof.

11. The solubilized benzoyl peroxide topical formulation of claim 7 that further comprises salicylic acid.

12. The solubilized benzoyl peroxide topical formulation of claim 7 that further comprises sodium hydroxide.

13. The use of the solubilized benzoyl peroxide topical formulation of claim 7 for the treatment and/or prophylaxis of acne.

14. A process for the preparation of a solubilized benzoyl peroxide topical formulation, said process comprising:
a. Combining one or more penetration enhancers, one or more solvents and surfactant at 25 to 40 ° C. with constant stirring;
b. Adding benzoyl peroxide powder to the foregoing solution; and
c. Cooling the resultant solution to room temperature.

15. The process of claim 14 wherein the penetration enhancer is dimethylsulfoxide, the surfactant is sodium lauryl sulfate, and the solvent is selected from: ethylene glycol-400 (low molecular weight solvent), propylene or butylene glycol, isopropyl or ethyl alcohol, short chain alkyl esters, ethers, aldehydes, ketones or alcohols of benzoic acid, benzyl alcohol, phenol or phathalic acid, aryl esters, ethers, alcohols of benzoic acid, benzyl alcohol, phenol, alkyl esters of benzoic acid, alkyl esters of benzyl alcohol, alkyl esters of salicylic acid, alkyl esters of phenol, alkyl esters of phthalic acid, alkyl ethers of benzyl alcohol, alkyl esters of phthalic acid, alkyl ethers of benzyl alcohol, alkyl ethers of phenol, benzoyl benzoate, benzoyl alcohol, diethyl phthalate, benzoic acid 2-phenyl ethyl ester, methyl salicylate, ethyl salicylate, propyl salicylate, butyl salicylate, ethyl benzoate, methyl benzoate, propyl benzoate, butyl benzoate, dimethyl phthalate, diethyl phthalate, benzyl ethyl ether, benzyl methyl ether, phenetole, phenyl acetone, phenyl ethyl alcohol, phenoxyethanol, phenyl acetaldehyde, ethyl phenyl acetate, phenyl methyl ketone, phenyl acetate, benzyl acetate, benzyl aceto acetate, benzyl formate, benzyl alcohol, ethyl benzyl alcohol, phenyl benzoate, phenyl ether, benzyl benzoate, and phenyl ethyl ester or combinations thereof.
